(19) United States
(12) Patent Application Publication
(10) Pub. No.: US 2007/0083094 A1
(43) Pub. Date: Apr. 12, 2007

(54) Medical sensor and technique for using the same

(76) Inventors: Joel C. Colburn, Walnut Creek, CA (US); Paul D. Mannheimer, Danville, CA (US); Michael P. O'Neil, Pleasanton, CA (US); Gilbert Hausmann, Felton, CA (US); Shannon E. Campbell, Oakland, CA (US)

Correspondence Address: FLETCHER YODER (TYCO INTERNATIONAL, LTD.), P.O. BOX 692289, HOUSTON, TX, 77269-2289, US

(21) Appl. No.: 11/369,361

(22) Filed: Mar. 7, 2006

Related U.S. Application Data
(60) Provisional application No. 60/725,466, filed on Oct. 11, 2005.

(51) Int. Cl. A61B 5/00

(52) U.S. Cl. 600/323

(57) ABSTRACT

A sensor is provided that is appropriate for transcutaneous detection of tissue or blood constituents. An electrochemical sensor for tissue constituent detection may include sensing materials that may be dry stored without liquid calibrant. The sensor may also include a temperature sensor that detects variations in tissue temperature at the sensor site. The tissue constituent measurements may be corrected in light of temperature variations of the tissue.

### CROSS-REFERENCE TO RELATED APPLICATIONS

This application claims priority to U.S. Provisional Application No. Ser. No. 60/725,466, filed Oct. 11, 2005, the disclosure of which is hereby incorporated by reference.

### BACKGROUND OF THE INVENTION

1. Field of the Invention

The present invention relates generally to medical devices and, more particularly, to sensors used for sensing physiological parameters of a patient.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

Physiological characteristics that physicians may desire to monitor include constituents of the blood and tissue, such as oxygen and carbon dioxide. For example, abnormal levels of carbon dioxide in the blood or tissue may be related to poor perfusion. Thus, assessment of carbon dioxide levels may be useful for diagnosing a variety of clinical states related to poor perfusion. Carbon dioxide and other blood constituents may be directly measured by taking a blood sample, or may be indirectly measured by assessing the concentration of those constituents in the tissue or respiratory gases. For example, carbon dioxide in the bloodstream equilibrates rapidly with carbon dioxide in the lungs, and the partial pressure of the carbon dioxide in the lungs approaches the amount in the blood during each breath. Accordingly, physicians often monitor respiratory gases during breathing in order to estimate the carbon dioxide levels in the blood.

However, estimation of carbon dioxide by respiratory gas analysis has certain disadvantages. It is often inconvenient to measure carbon dioxide in respiratory gases from respiratory gas samples collected from an endotracheal tube or cannula. Although these methods are considered to be noninvasive, as the surface of the skin is not breached, the insertion of such devices may cause discomfort for the patient. Further, the insertion and operation of such devices also involves the assistance of skilled medical personnel.

Carbon dioxide in the tissue and in certain cases carbon dioxide in the blood that diffuses into the tissue may also be measured transcutaneously by a sensor or sensors placed against a patient's skin. While or sensors are easier to use than respiratory gas sensors, they also have certain disadvantages. Such sensors may employ optical, chemical, or electrochemical carbon dioxide indicators, and such sensors typically are stored in calibration fluid prior to use. Although the calibration fluid may improve measurement accuracy, the use of calibration fluid presents storage, transportation, and cost challenges for such sensors.

Thus, it may be desirable to provide a transcutaneous sensor for the measurement of carbon dioxide and other tissue or blood gases or other components that may not require a liquid storage medium and which does not cause discomfort for the patient.

### SUMMARY

Certain aspects commensurate in scope with the originally claimed invention are set forth below. It should be understood that these aspects are presented merely to provide the reader with a brief summary of certain forms that the invention might take, and that these aspects are not intended to limit the scope of the invention. Indeed, the invention may encompass a variety of aspects that may not be set forth below.

There is provided a sensor that includes: a non-optical transducer, wherein the non-optical transducer is adapted to provide an electrical signal related to a tissue constituent; and a gas collection chamber.

There is provided a system that includes: a monitor; and a sensor adapted to be operatively coupled to the monitor, the sensor including: a non-optical transducer, wherein the non-optical electrochemical transducer is adapted to provide an electrical signal related to a tissue constituent; and a gas collection chamber.

There is provided a method that includes: contacting a tissue constituent collected in a gas collection chamber with a non-optical transducer, wherein the non-optical transducer is adapted to provide an electrical signal related to the tissue constituent.

There is provided a method that includes: providing a sensor body comprising a gas collection chamber; and disposing a non-optical transducer on the sensor body, wherein the non-optical transducer is adapted to provide an electrical signal related to a tissue constituent.

There is provided a sensor system that includes: at least one sensor, the sensor including: a sensor body comprising a gas collection chamber; and a non-optical transducer layer disposed on the sensor body, wherein the non-optical transducer is adapted to provide a signal related to a tissue constituent.

There is provided a sensor that includes: a sensor body comprising a gas collection chamber adapted to be placed against a patient's tissue; a transducer disposed on the sensor body adapted to provide signal related to a tissue constituent; and a temperature sensor disposed on the sensor body adapted to provide signal related to the temperature of the patient's tissue.

There is provided a system that includes: a monitor; and a sensor adapted to be operatively coupled to the monitor, the sensor including: a sensor body comprising a gas collection chamber adapted to be placed against a patient's tissue; a transducer disposed on the sensor body adapted to provide signal related to a tissue constituent; and a temperature sensor disposed on the sensor body adapted to provide signal related to the temperature of the patient's tissue.

There is provided a method that includes: acquiring gas data related to a gas content of a tissue; acquiring temperature data related to a temperature of the tissue; obtaining a correction factor based on the temperature data; and calculating temperature-corrected gas data based on the gas data and the correction factor.

There is provided a method that includes: providing a sensor body comprising a gas collection chamber adapted to be placed against a patient's tissue; providing a transducer disposed on the sensor body adapted to provide signal related to a tissue constituent; and providing a temperature sensor disposed on the sensor body adapted to provide signal related to the temperature of the patient's tissue.

There is provided a sensor that includes: a sensor body adapted to form a gas collection chamber when placed against a patient's tissue; an electrochemical transducer disposed on the sensor body, wherein the electrochemical transducer is adapted to change its electrical properties in response to the presence of carbon dioxide; and a cable electrically coupled to the electrochemical transducer.

There is provided a sensor that includes: a sensor body adapted to be placed against a patient's tissue; and a transducer-utilizing quantum-restricted or semi-conductive material that is disposed on the sensor body, wherein a property of the quantum-restricted or semi-conductive material is affected by the presence of an analyte.

### BRIEF DESCRIPTION OF THE DRAWINGS

Advantages of the invention may become apparent upon reading the following detailed description and upon reference to the drawings in which:

FIG. 1 is a schematic cross-section of a sensor showing a non-optical transducer adapted to provide an electrical response according to the present invention;

FIG. 2 illustrates a perspective view of a patient using a sensor for detection of a physiological constituent according to the present invention;

FIG. 3 illustrates a cross-sectional view of a sensor for detection of tissue or blood constituents with a collection chamber and a non-optical transducer adapted to provide an electrical feedback according to the present invention;

FIG. 4 illustrates a cross-sectional view of a sensor for detection of tissue or blood constituents with a non-optical transducer adapted to provide an electrical feedback and a selective barrier that has been disposed on the non-optical transducer according to the present invention;

FIG. 5 illustrates a cross-sectional view of a sensor for detection of tissue or blood constituents with a non-optical transducer adapted to provide an electrical feedback and a temperature sensor according to the present invention;

FIG. 6 is a flow chart of a data correction process dependent on temperature according to the present invention;

FIG. 7 illustrates a cross-sectional view of a sensor without a gas collection chamber for detection of tissue or blood constituents with a semi-conductive or quantum-restricted transducer adapted to provide an electrical feedback and a temperature sensor according to the present invention;

FIG. 8 illustrates a semi-dry or dry storage system with a protective package for a sensor according to the present techniques; and

FIG. 9 illustrates a physiological constituent detection system coupled to a multi-parameter patient monitor and a sensor according to embodiments of the present invention.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A sensor is provided herein that may assess a tissue constituent, such as a tissue gas or substance (such as oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic) with a non-optical transducer that is adapted to provide an electrical signal. Such a sensor provides cost and convenience advantages. Sensors according to the present techniques may be stored without calibration fluid or other liquids, as the non-optical sensor may maintain its calibration state in dry and/or semi-dry storage. Thus, a sensor may be stored without the need for a healthcare worker to maintain calibration fluid levels in the storage system to prevent drying out of the sensor. Further, as the sensor maintains its calibration state for longer periods of time, the sensor need not be calibrated before every use.

Sensors according to the present techniques may transcutaneously sense carbon dioxide or other tissue constituents in a tissue layer and transduce an electrical feedback. For example, carbon dioxide and other constituents in the bloodstream may diffuse through the tissue and may dissolve into any liquids that may be found at the surface of the tissue. Thus, the levels of carbon dioxide or other constituents in the tissue may serve as a surrogate marker for carbon dioxide levels in the bloodstream. A sensor according to the present techniques placed proximate to a tissue surface may capture and measure carbon dioxide that would otherwise diffuse into the airstream or other surrounding airspace.

Generally, it is envisioned that a sensor according to the present technique is appropriate for use in determining the presence or levels of tissue constituents in a variety of tissues. The sensor may be held against the tissue, either manually, mechanically, adhesively, or otherwise, for the purpose of forming a seal to prevent the carbon dioxide from diffusing away. For example, a sensor may be used in the upper respiratory tract, including the oral and nasal passages. The oral passages may include the tongue, the floor of the mouth, the roof of the mouth, the soft palate, the cheeks, the gums, the lips, and any other oral tissue. Further, a sensor as described herein is appropriate for use adjacent to or proximate to any mucosal surface, i.e. patient surfaces that include a mucous membrane or surfaces that are associated with mucus production. In addition to the respiratory tract, mucosal surfaces may include vaginal or rectal surfaces.

Sensors as provided by the present techniques may be disposable or reusable. In addition, the sensors may be appropriate for short-term spot-checking or for longer-term, continuous monitoring. When used for long-term monitoring, the sensor may be applied to the patient's tissue by a suitable adhesive, such as a mucoadhesive, or by any other suitable holding device.

In addition to carbon dioxide monitoring, sensors as provided herein may be used to monitor oxygen, carbon monoxide, volatile organic compounds such as ethanol, metabolic trace gases such as acetone or anesthetic gases such as isoflurane, halothane, desflurane, sevoflurane and enflurane that may diffuse transcutaneously. In certain embodiments, it may be useful to measure concentration of a tissue constituent and compare the tissue concentration to a normal blood concentration or a blood concentration obtained by direct measurement of a blood sample. For example, sensors as provided herein may be used to monitor tissue gases associated with an acute or chronic disease state. Such sensors may monitor hydrogen ions or bicarbonate ions in the tissue as a marker to assess the acidity of the blood. Variations from normal blood pH may be useful in assessing medical conditions.

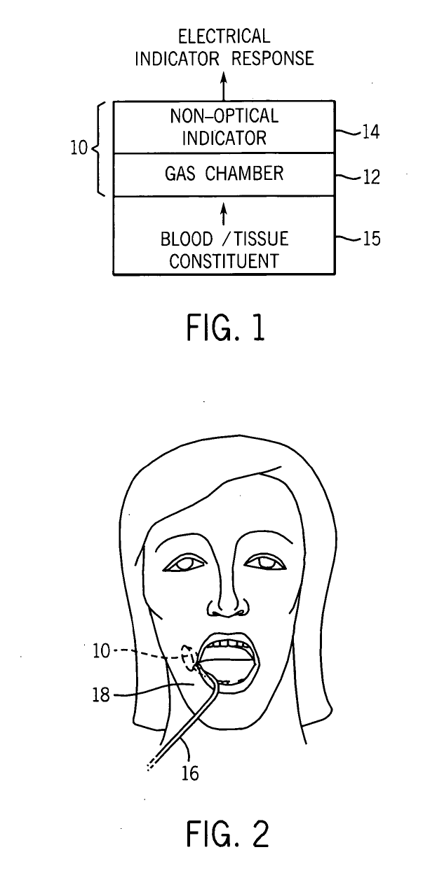

FIG. 1 is a schematic view of an exemplary sensor 10. The sensor 10 has a gas collection chamber 12 and a non-optical transducer 14. When the sensor 10 is contacted with a tissue sensor site, blood or tissue constituents 15 perfuse through the tissue and enter the collection chamber 12. The non-optical transducer 14 is adapted to respond to the presence of the blood or tissue constituents 15, and to provide an electrical feedback, as discussed in more detail below. The non-optical transducer 14 is sensitive to the presence of a tissue constituent and may be capable of being calibrated to give an electrical response signal corresponding to a given predetermined concentration of the tissue constituent. In certain embodiments, the electrical feedback may be related to the concentration of the tissue constituent, or the partial pressure of the tissue constituent.

The non-optical transducer 14 may be an electrochemical transducer, which may be adapted to detect and measure changes in ambient chemical parameters induced by the presence of critical amounts of a tissue constituent. In one embodiment, the non-optical transducer 14 may include a sensor that employs cyclic voltammetry for carbon dioxide detection. Such sensors are available from Giner, Inc., Newton, Mass. For example, the non-optical transducer 14 may be a thick film catalyst sensor utilizing a proton exchange membrane. Such a non-optical transducer 14 may include thick film screen printed electrodes and an electrochemically reversible metal oxide catalysts. Appropriate catalysts include MO, M₂O₃, MO₂, where M is a metal that is any suitable metal, including platinum ruthenium or iridium. Generally, such sensors operate by sensing chemical reactions caused by proton dissociation from water in which carbon dioxide is dissolved. Dissociated water protons may electrochemically reduce a metal oxide layer of the sensor. The electrochemical reduction of the metal oxide will result in generation of an electrical current, which varies in response to the degree of electrochemical reduction.

In another embodiment, the non-optical transducer 14 may include quantum-restricted components, including carbon nanotubes, buckeyballs, or quantum dots. Generally, quantum-restricted components may be coated or otherwise modified with a compound that is sensitive to the tissue constituent of interest. Interaction of the tissue constituent with the compound may affect the electrical, optical, thermal, or physical properties of the quantum-restricted components such that a signal may result. In one such example, carbon nanotubes may be coated with a carbon dioxide-sensitive compound or polymer, such as a polyethyleneimine and starch polymer. Carbon dioxide may combine with primary and tertiary amines in the polyethyleneimine and starch polymer coating to form carbamates. The chemical reaction alters the charge transfer to the carbon nanotube and resulting in an electrical signal of the transducer. Other suitable polymer coatings may be adapted to sense other tissue constituents of interest, such as oxygen or carbon monoxide. In other embodiments, the quantum-restricted component may include a binding molecule, such as a receptor or an enzyme that is specific for the tissue constituent of interest. One such molecule may include carbonic anhydrase. Binding of the tissue constituent to its receptor may affect a downstream response that may result in a change in the electrical properties of a quantum-restricted component.

The sensing component may also include a semi-conductive sensing element, such as a field-effect transistor (FET) or an ion-sensitive field-effect transistor (ISFET). An ISFET may include a silicon dioxide gate for a pH selective membrane. Such a sensor may be adapted to sense downstream changes in hydrogen ion concentration in response to changes in carbon dioxide or other tissue constituent concentrations. In certain embodiments, the semi-conductive sensing element may be a film.

In specific embodiments, it may be advantageous to provide a sensor for in vivo use on a patient's buccal or sublingual tissue that is easily reached by the patient or a healthcare worker. For example, FIG. 2 illustrates the placement of a sensor on a buccal surface of a patient in order to assess a tissue gas, for example carbon dioxide, in the tissue, blood or interstitial fluid. Specifically, FIG. 2 shows an embodiment of a sensor 10 including a conduit 16 in communication with the sensor 10. In certain embodiments, the conduit 16 may be adapted to transmit an electrical feedback from the sensor 10 to a monitor. In another embodiment, the conduit 16 may be adapted to transport gases from the sensor 10. In such an embodiment, the sensor 10 may collect tissue gases in a chamber. The collected gases may then diffuse through the conduit 16 that is connected to the collection chamber, and the gases may then be further assessed and/or measured by sensing elements not directly applied to the patient. The sensor 10 may be suitably sized and shaped such that a patient may easily close his or her mouth around the sensor with minimal discomfort.

The sensor 10 is secured to the patient's buccal tissue 18 such that the area covered by the sensor 10 is substantially sealed to prevent gas flow in or out of the sensor 10, thus preventing tissue gases at the sensor placement site from dissipating into the air stream or escaping out of the air stream, which may lead to inaccurate measurements. Further, the sensor's 10 tissue seal may also prevent respiratory gases or oral fluids from entering the sensor 10. Generally, the sensor 10 may be suitably sized and shaped to allow the sensor 10 to be positioned near or flush against the buccal tissue 18.

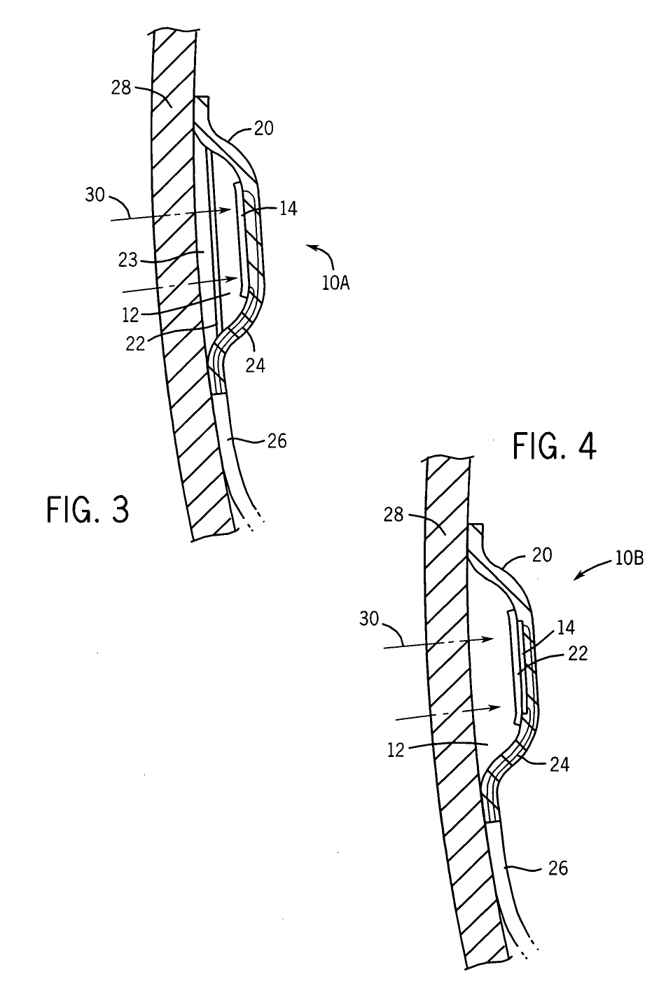

FIG. 3 is a cross-sectional view of an exemplary sensor 10A held against a mucosal tissue 28. The sensor 10A includes a housing 20 surrounding a non-optical transducer 14. The housing is formed to provide a surface that is suitably shaped to be secured against a mucosal tissue. The housing 20 may be any suitable material that is generally suited to the aqueous environment of the mucous membrane. For example, the housing 20 may be formed from: a metal, polypropylene, polyethylene, polysulfone or similar polymers. Generally, the housing should be relatively impermeable to tissue constituents 30, such that the sensor 10A may collect tissue constituents 30, such as tissue gases, for a sufficient period of time to allow for detection and measurement. Hence, it may be advantageous to coat the sensor 10A with additional sealants to prevent leakage of the tissue constituents 30. The housing 20, once secured to the tissue, forms a collection chamber 12 that traps tissue constituents 30 that diffuse through the mucosal tissue 28. The trapped tissue gas 30 may then be sensed by the non-optical transducer 14, which is electrically coupled to a cable 26 by a wire or wires 24 in order to provide an electrical signal. It is envisioned that the volume of the collection chamber 12 may be optimized to be large enough to allow sufficient tissue constituents 30 to be collected while being small enough to provide rapid response times.

In certain embodiments, the sensor 10A may include materials that function as a selective barrier 22 that are hydrophobic or otherwise water-resistant, but are permeable to carbon dioxide or other constituent gases. For example, a selective barrier 22 may form a tissue contact surface of the sensor 10A that prevents water from entering the sensor 10A. In such an embodiment, carbon dioxide in the tissue would perfuse through the contact surface to enter the gas collection chamber 12. In one embodiment, it is envisioned that the ratio of water permeability to carbon dioxide permeability of a selective barrier 22 may be less than 10, and in certain embodiments, the ratio may be less than 1. Suitable materials include polymers, such as polytetrafluorethylene (PTFE). Other suitable materials include microporous polymer films, such as those available from the Landec Corporation (Menlo Park, Calif.). Such microporous polymer films are formed from a polymer film base with a customizable crystalline polymeric coating that may be customized to be highly permeable to carbon dioxide and relatively impermeable to water. The thickness of a selective barrier 22 may be modified in order to achieve the desired rate of carbon dioxide perfusion and transducer response time. Generally, response times may be in the range of instantaneous to less than 5 minutes. In certain embodiments, the response time is in the range of 5 seconds to 5 minutes. Where a very rapid response is desired, a thin film of the selective barrier 22, for example less than 0.2 mm in thickness, may be used. In certain embodiments, when a slower response is desired, a selective barrier 22 may range from 0.2 mm to several millimeters in thickness. Additionally, the selective barrier 22 may be formed with small pores that increase the carbon dioxide permeability. The pores may be of a size of 0.01 to approximately 10 microns, depending on the desired response time. In one embodiment, the selective barrier 22 may be a relatively thin PTFE material such as plumber's tape (0.04 mm). In other embodiments, the selective barrier 22 may be a PTFE material such as Gore-Tex® (W. L. Gore & Associates, Inc., Newark, Del.). Alternatively, the selective barrier 22 may be formed from a combination of appropriate materials, such as materials that are heat-sealed or laminated to one another. For example, the selective barrier 22 may include a PTFE layer with a pore size of 3 microns and a second PTFE layer with a pore size of 0.1 microns.

Additionally, in certain embodiments, a sensor 10A may also include a porous substrate 23 which is permeable to a wide variety of tissue constituents. As a selective barrier 22 may be quite thin, the porous substrate 23 may be advantageous in providing rigidity and support to the sensor 10A. Suitable materials include paper, plastics, inorganic, glassy, or woven materials.

In certain embodiments, as shown in FIG. 4, a sensor 10B may include a selective barrier 22 that is directly applied to the non-optical transducer 14. Thus, the gas collection chamber 12 may allow water vapor to diffuse in from the tissue 28. However, such water vapor is prevented from interfering with the sensing components by the selective barrier 22. The selective barrier 22 may be applied to the non-optical transducer 14 by plasma deposition or screen printing.

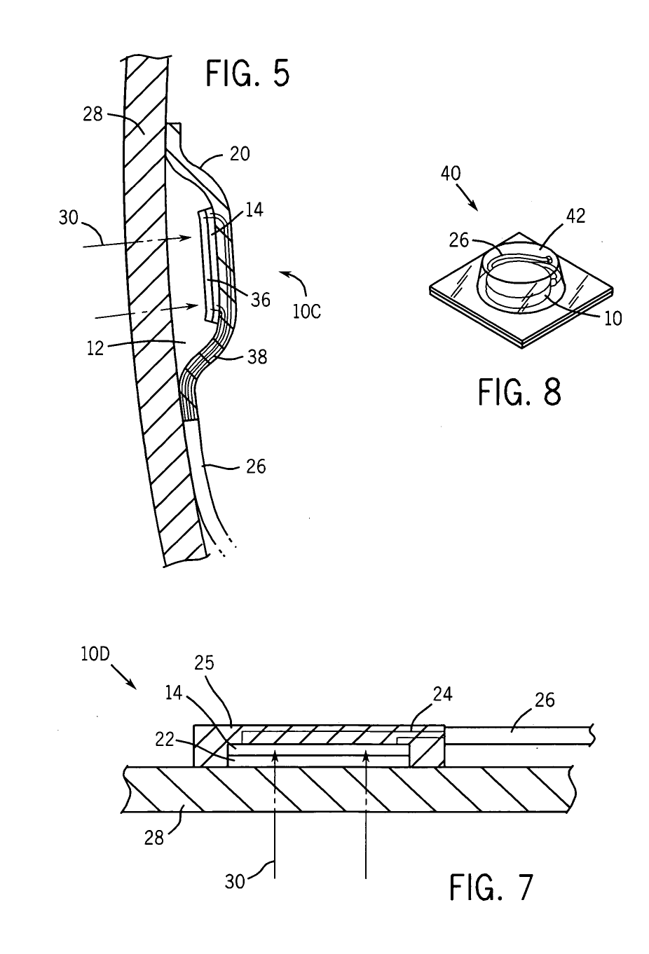

FIG. 5 is a cross-sectional sensor view of a 10C that includes a temperature sensor 36 disposed on or proximate to a non-optical transducer 14. Such an arrangement may be advantageous when the non-optical transducer 14 has strong temperature dependence in its feedback. As depicted, feedback for both the non-optical transducer 14 and the temperature sensor 36 may be obtained by electrically coupling the non-optical transducer 14 and the temperature sensor 36 to a cable 26 by a series of wires 38. In the embodiment shown in FIG. 5, the temperature sensor 36 may be applied to the non-optical transducer 14 by a thick film deposition technique.

In other embodiments (not shown), a temperature sensor 36 may contact the tissue surface. Other suitable temperature sensors 36 according to the present techniques include any suitable medical grade temperature sensor, such as resistance-based temperature sensors and infrared temperature sensors available from Thermometrics (Plainville, Conn.). A sensor 10C may include multiple temperature sensors 36.

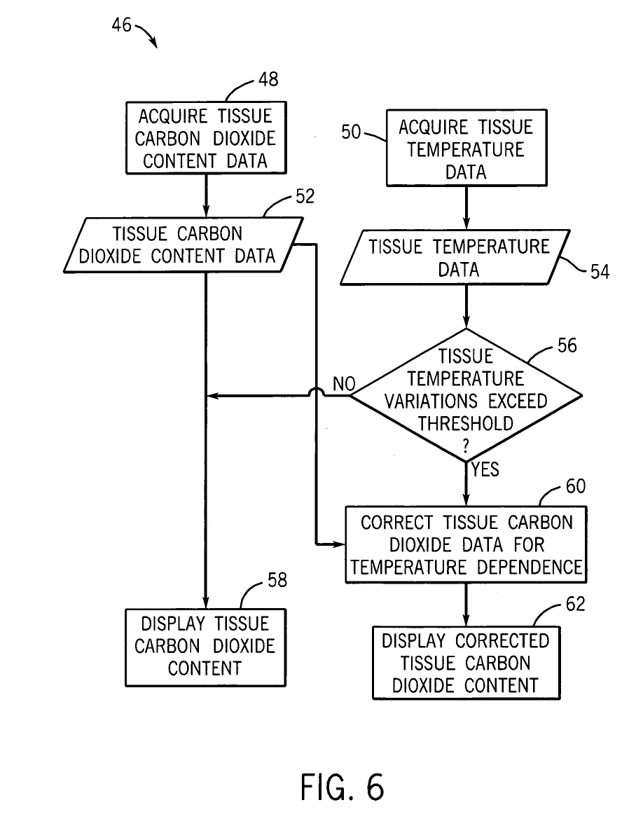

It is envisioned that a temperature sensor 36 as described herein may be used to provide information related to the temperature at the sensor 10 measurement site during use. Such information may be converted into an electrical signal and sent to a monitor or another appropriate device, as described in more detail below, for processing. The flow chart 46 depicted in FIG. 6 describes the downstream steps involved after step 48, which involves acquisition of tissue carbon dioxide data 52 from the sensor 10, and step 50, which involves acquisition of tissue temperature data 54. It should be understood that the data related to the tissue concentration of any contemplated tissue constituent may be acquired at step 48, and that carbon dioxide is merely used as an illustrative example. In certain embodiments, it is envisioned that steps 48 and 50 may occur simultaneously.

At a step 56, a processor analyzes the tissue temperature data 54 to determine if the tissue temperature data 54 may be associated with a temperature-dependent artifact or measurement error. For example, certain variations in the tissue temperature, as directly measured on the tissue or as indirectly measured in a tissue gas collection chamber, may influence the signal of an electrochemical transducer. If the temperature data 54 is indicative of a likelihood of a signal error, a processor passes control to step 60. Generally, the tissue temperature data 54 outputs from a temperature sensor 36 as described herein may be further acted upon by a processor to obtain a temperature correction factor. The temperature correction factor may then be applied at step 60 to the tissue carbon dioxide content data 52 in order to obtain corrected tissue carbon dioxide content. The temperature-corrected tissue carbon dioxide content may be displayed on a monitor at step 62.

If, at a step 56, the tissue temperature data does not exceed a predetermined threshold value or a predetermined likelihood of being associated with a signal error, the processor passes control to step 58. At step 58 the system displays tissue carbon dioxide content on a monitor after the system goes into a default mode and a processor calculates a tissue carbon dioxide content from the tissue carbon dioxide content data 52.

In other embodiments, it may be advantageous to provide a sensor 10D, as depicted in FIG. 7, with a compact design and housing 25 that is generally flat to easily fit inside the mouth of other tissue of a user. Such a sensor 10D need not include any gas collection area, as the tissue constituent 30 may diffuse directly into a non-optical transducer 14 from the tissue 28. A sensor 10D may also include an optional barrier layer 22 to prevent water from damaging the non-optical transducer. The non-optical transducer may communicate through wires or electrical leads 24 and a cable 26 with a patient monitor.

In certain embodiments, the present techniques provide a dry storage system 40 shown in FIG. 8 for the exemplary sensors 10 described herein. For example, it may be advantageous to package the sensor 10 in foil, plastic, or other protective materials in order to protect the sensor 10 from exposure to environmental damage during transportation and prior to use. A dry storage system 40 may include a protective package 42, such as a blister package. As the non-optical transducer 14 need not be packaged in calibration fluid, the protective package 42 may be vacuum-sealed, or may contain an inert gas. In certain embodiments, the sensor 10 may be packaged with all or part of a cable 26.

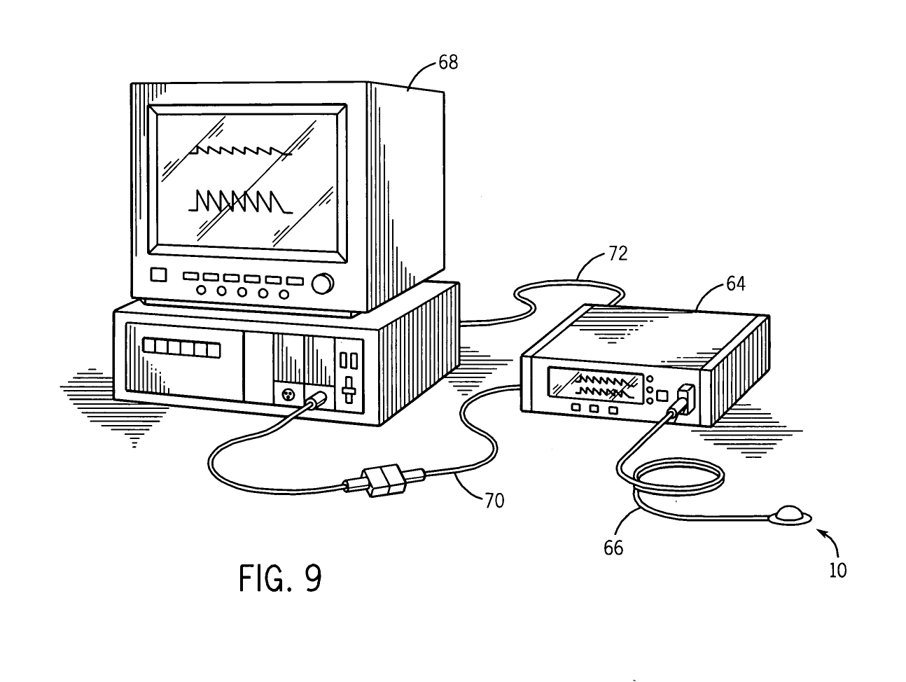

The exemplary sensors described herein, described here generically as a sensor 10, may be coupled to a monitor 64 that may display the concentration of tissue constituents as shown in FIG. 9. It should be appreciated that the cable 66 of the sensor 10 may be coupled to the monitor 64 or it may be coupled to a transmission device (not shown) to facilitate wireless transmission between the sensor 10 and the monitor 64. Furthermore, to upgrade conventional tissue constituent detection provided by the monitor 64 to provide additional functions, the monitor 64 may be coupled to a multi-parameter patient monitor 68 via a cable 70 connected to a sensor input port or via a cable 72 connected to a digital communication port.

While the invention may be susceptible to various modifications and alternative forms, specific embodiments have been shown by way of example in the drawings and have been described in detail herein. However, it should be understood that the invention is not intended to be limited to the particular forms disclosed. Indeed, the present techniques may not only be applied to measurements of carbon dioxide, but these techniques may also be utilized for the measurement and/or analysis of other tissue and/or blood constituents. Rather, the invention is to cover all modifications, equivalents, and alternatives falling within the spirit and scope of the invention as defined by the following appended claims. It will be appreciated by those working in the art that sensors fabricated using the presently disclosed and claimed techniques may be used in a wide variety of contexts. That is, while the invention has primarily been described in conjunction with the measurement of carbon dioxide concentration in blood, the sensors fabricated using the present method may be used to evaluate any number of sample types in a variety of industries, including fermentation technology, cell culture, and other biotechnology applications.

What is claimed is:

1. A sensor comprising:
a non-optical transducer, wherein the non-optical transducer is adapted to provide an electrical signal related to a tissue constituent; and
a gas collection chamber.

2. The sensor, as set forth in claim 1, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, a volatile organic compound, a chemical warfare agent, or a narcotic.

3. The sensor, as set forth in claim 1, comprising a selective barrier disposed on the sensor body that is substantially impermeable to water.

4. The sensor, as set forth in claim 3, wherein the selective barrier is disposed on a surface of the non-optical transducer.

5. The sensor, as set forth in claim 1, comprising a temperature sensor adapted to provide signal related to a tissue temperature.

6. The sensor, as set forth in claim 1, wherein the non-optical transducer comprises an electrochemical transducer.

7. The sensor, as set forth in claim 1, wherein the non-optical transducer comprises a metal oxide.

8. The sensor, as set forth in claim 1, wherein the non-optical transducer comprises a quantum-restricted element.

9. A system comprising:
a monitor; and
a sensor adapted to be operatively coupled to the monitor, the sensor comprising:
a non-optical transducer, wherein the non-optical electrochemical transducer is adapted to provide an electrical signal related to a tissue constituent; and a gas collection chamber.

10. The system, as set forth in claim 9, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

11. The system, as set forth in claim 9, comprising a selective barrier disposed on the sensor body that is substantially impermeable to water.

12. The system, as set forth in claim 11, wherein the selective barrier is disposed on a surface of the non-optical transducer.

13. The system, as set forth in claim 9, comprising a temperature sensor adapted to provide signal related to a tissue temperature.

14. The system, as set forth in claim 9, wherein the non-optical transducer comprises an electrochemical transducer.

15. The system, as set forth in claim 9, wherein the non-optical transducer comprises a metal oxide.

16. The system, as set forth in claim 9, wherein the non-optical transducer comprises a quantum-restricted element.

17. The system, as set forth in claim 9, comprising a multi-parameter monitor.

18. A method comprising:
contacting a tissue constituent collected in a gas collection chamber with a non-optical transducer, wherein the non-optical transducer is adapted to provide an electrical signal related to the tissue constituent.

19. The method, as set forth in claim 18, comprising contacting the tissue constituent with a selective barrier disposed that is substantially impermeable to water.

20. The method, as set forth in claim 18, comprising contacting a tissue or tissue constituent with a temperature sensor adapted to provide signal related to the tissue temperature.

21. The method, as set forth in claim 18, wherein the non-optical transducer comprises a quantum-restricted element.

22. A method of manufacturing a sensor, comprising:
providing a sensor body comprising a gas collection chamber; and
disposing a non-optical transducer on the sensor body, wherein the non-optical transducer is adapted to provide an electrical signal related to a tissue constituent.

23. The method, as set forth in claim 22, wherein the tissue constituent comprises carbon dioxide or carbon monoxide.

24. The method, as set forth in claim 22, wherein the tissue constituent comprises oxygen.

25. The method, as set forth in claim 22, comprising a selective barrier disposed on the sensor body that is substantially impermeable to water.

26. The method, as set forth in claim 22, comprising a temperature sensor adapted to provide signal related to a tissue temperature.

27. The method, as set forth in claim 22, wherein the non-optical transducer comprises an electrochemical transducer.

28. The method, as set forth in claim 22, wherein the non-optical transducer comprises a metal oxide.

29. The method, as set forth in claim 22, wherein the non-optical transducer comprises a quantum-restricted element

30. A sensor system comprising:
at least one sensor, the sensor comprising:
a sensor body comprising a gas collection chamber; and
a non-optical transducer layer disposed on the sensor body, wherein the non-optical transducer is adapted to provide a signal related to a tissue constituent.

31. The sensor system, as set forth in claim 30, comprising a protective package enclosing the sensor, wherein the protective package does not include calibration fluid.

32. The sensor system, as set forth in claim 30, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

33. The sensor system, as set forth in claim 30, comprising a selective barrier disposed on the sensor body that is substantially impermeable to water.

34. The sensor system, as set forth in claim 33, wherein the selective barrier is disposed on a surface of the non-optical transducer.

35. The sensor system, as set forth in claim 30, comprising a temperature sensor adapted to provide signal related to a tissue temperature.

36. The sensor system, as set forth in claim 30, wherein the non-optical transducer comprises an electrochemical transducer.

37. The sensor system, as set forth in claim 30, wherein the non-optical transducer comprises a metal oxide.

38. The sensor system, as set forth in claim 30, wherein the non-optical transducer comprises a quantum-restricted element.

39. The sensor system, as set forth in claim 30, wherein the signal comprises an electrical signal.

40. A sensor comprising:
a sensor body comprising a gas collection chamber adapted to be placed against a patient's tissue;
a transducer disposed on the sensor body adapted to provide signal related to a tissue constituent; and
a temperature sensor disposed on the sensor body adapted to provide signal related to the temperature of the patient's tissue.

41. The sensor, as set forth in claim 40, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

42. The sensor, as set forth in claim 40, comprising a selective barrier disposed on the sensor body that is substantially impermeable to water.

43. The sensor, as set forth in claim 40, wherein the selective barrier is disposed on a surface of the non-optical transducer.

44. The sensor, as set forth in claim 40, wherein the temperature sensor is disposed on the transducer.

45. The sensor, as set forth in claim 40, wherein the transducer comprises an electrochemical transducer.

46. The sensor, as set forth in claim 40, wherein the transducer comprises a metal oxide.

47. The sensor, as set forth in claim 40, wherein the non-optical transducer comprises a quantum-restricted element.

48. A system comprising:
a monitor; and
a sensor adapted to be operatively coupled to the monitor, the sensor comprising:
a sensor body comprising a gas collection chamber adapted to be placed against a patient's tissue;
a transducer disposed on the sensor body adapted to provide signal related to a tissue constituent; and
a temperature sensor disposed on the sensor body adapted to provide signal related to the temperature of the patient's tissue.

49. The system, as set forth in claim 48, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

50. The system, as set forth in claim 48, comprising a selective barrier disposed on the sensor body that is substantially impermeable to water.

51. The system, as set forth in claim 48, wherein the selective barrier is disposed on a surface of the non-optical transducer.

52. The system, as set forth in claim 48, wherein the temperature sensor is disposed on the transducer.

53. The system, as set forth in claim 48, wherein the transducer comprises an electrochemical transducer.

54. The system, as set forth in claim 48, wherein the transducer comprises a metal oxide.

55. The system, as set forth in claim 48, wherein the non-optical transducer comprises a quantum-restricted element.

56. The system, as set forth in claim 48, comprising a multi-parameter monitor.

57. A method comprising:
acquiring gas data related to a gas content of a tissue;
acquiring temperature data related to a temperature of the tissue;
obtaining a correction factor based on the temperature data; and
calculating temperature-corrected gas data based on the gas data and the correction factor.

58. The method, as set forth in claim 57, comprising displaying the temperature-corrected gas data.

59. A method of manufacturing a sensor, comprising:
providing a sensor body comprising a gas collection chamber adapted to be placed against a patient's tissue;
providing a transducer disposed on the sensor body adapted to provide signal related to a tissue constituent; and
providing a temperature sensor disposed on the sensor body adapted to provide signal related to the temperature of the patient's tissue.

60. The method, as set forth in claim 59, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

61. The method, as set forth in claim 59, comprising a selective barrier disposed on the sensor body that is substantially impermeable to water.

62. The method, as set forth in claim 59, wherein the temperature sensor is disposed on the transducer.

63. The method, as set forth in claim 59, wherein the transducer comprises an electrochemical transducer.

64. The method, as set forth in claim 59, wherein the electrochemical transducer comprises a metal oxide.

65. The method, as set forth in claim 59, wherein the non-optical transducer comprises a quantum-restricted element.

66. A sensor comprising:
a sensor body adapted to form a gas collection chamber when placed against a patient's tissue;
an electrochemical transducer disposed on the sensor body, wherein the electrochemical transducer is adapted to change its electrical properties in response to the presence of carbon dioxide; and
a cable electrically coupled to the electrochemical transducer.

67. A sensor comprising:
a sensor body adapted to be placed against a patient's tissue; and
a quantum-restricted or semi-conductive transducer disposed on the sensor body, wherein the quantum-restricted or semi-conductive transducer is adapted to change its electrical properties in response to the presence of a tissue constituent.

68. The sensor, as set forth in claim 67, wherein the tissue constituent comprises oxygen, carbon dioxide, carbon monoxide, nitric oxide, nitrous oxide, helium, nitrogen, halothane, isoflurane, flurane, desflurane, sevoflurane, hydrocarbon 24, xenon, an anesthetic agent, amyl nitrite, acetone, ammonia, short-chain alkanes, propofol, dialdehydes, diazepam, lorazepam, midazolam, fentanyl, volatile organic compounds, a chemical warfare agent, or a narcotic.

69. The sensor, as set forth in claim 67, comprising a selective barrier disposed on the sensor body that is substantially impermeable to water.

70. The sensor, as set forth in claim 69, wherein the selective barrier is disposed on a surface of the non-optical transducer.
